(19) United States
(12) Patent Application Publication
(10) Pub. No.: US 2020/0239932 A1
(43) Pub. Date: Jul. 30, 2020

(54) EFFICIENT SCREENING LIBRARY PREPARATION

(71) Applicant: The Regents of the University of California, Oakland, CA (US)

(72) Inventors: Eric Van Nostrand, La Jolla, CA (US); Eugene Yeo, La Jolla, CA (US); Alexander Shishkin, La Jolla, CA (US)

(21) Appl. No.: 16/756,320

(22) PCT Filed: Oct. 15, 2018
(86) PCT No.: PCT/US2018/000383

§ 371 Date: Apr. 15, 2020

Related U.S. Application Data
(60) Provisional application No. 62/573,061, filed on Oct. 16, 2017.

(51) Int. Cl. C12Q 1/6806; C12Q 1/682; C12Q 1/6874

(52) U.S. Cl. CPC C12Q 1/6806; C12Q 1/6874; C12Q 1/682

(57) ABSTRACT

Described herein are efficient methods for preparing a library for use in comprising performing gene targeting or massively parallel reporter assays. The methods comprise performing hybrid capture of a library constant region.

### CROSS REFERENCE TO RELATED APPLICATIONS

This application claims priority under 35 U.S.C. § 119(e) to U.S. Provisional Application No. 62/573,061, filed Oct. 16, 2017, the content of which is hereby incorporated by reference in its entirety.

### STATEMENT OF GOVERNMENT SUPPORT

This invention was made with government support under Grant No. NS075449 awarded by the National Institutes of Health. The government has certain rights in the invention.

### BACKGROUND

There is great interest in performing pooled screens for a variety of purposes, including identifying drug resistance and delivery mechanisms, genes essential for survival, death and disease phenotypes, differentiation, regulation of gene expression, and various other mechanisms. High throughput screening libraries are a tool to provide genome-wide functional characterization of genetic elements in normal biological processes and disease.

One type of screening library is a targeted or genome-wide loss-of-function screen, designed to provide information about all annotated genes in a genome by knocking out every gene or knocking down every transcribed RNA. Traditional loss-of-function genetic screening is performed using RNA interference (RNAi), particularly in mammalian cells. However, RNAi has inherent limitations due to its tendency to produce off-target effects and incomplete knockdown of protein expression. Another type of screening library is a functional screen, designed to provide information about the function of sequence elements. Also referred to as “massively parallel reporter assays,” these typically take the form of sequences (either random or based on known genomic sequences) placed in the context of a reporter (typically fluorescence-based) that reads out the regulatory activity of the sequence under question.

Modern methods for generating high throughput sequencing libraries utilize a PCR amplification based strategy. However, the PCR amplification method is also highly inefficient, especially for large amounts of genomic DNA. In particular, a high concentration of genomic DNA will inhibit PCR and a low concentration of genomic DNA will incur a large handling cost.

Therefore, a need exists for more efficient and effective methods of preparing high-throughput screening libraries. This disclosure satisfies this need and provides related advantages as well.

### SUMMARY

The methods described herein enable generation of high throughput sequencing libraries from DNA isolated from a population of cells containing a pooled library (e.g., a pooled gene targeting library). After genomic DNA isolation, a hybrid capture is performed using antisense RNA probes specifically recognizing the integrated DNA fragment. By washing away unrelated genomic DNA, PCR amplification of desired fragments is dramatically improved for identification by high throughput sequencing. These methods significantly improve efficiency of library preparation, increasing signal-to noise ratio in identifying true targets. Importantly, Applicant's methods are platform and library agnostic, and provide a dramatic improvement for all such approaches by simplifying and improving library preparation, enabling larger scale studies, higher reproducibility, and higher sensitivity in identifying candidates for further study.

Accordingly, in some aspects, provided herein are methods of preparing a pooled library, the methods comprising, consisting of, or consisting essentially of: (a) performing hybrid capture of nucleic acids in a sample comprising a pooled library; (b) isolating the captured nucleic acids; and (c) amplifying the isolated, captured nucleic acids. In some embodiments, the methods further comprise, consist of, or consist essentially of (d) performing high throughput sequencing analysis of the amplified nucleic acids produced in step (c). In some embodiments, the pooled library is a gene targeting library. In some embodiments, the pooled library is a reporter library for massively parallel reporter assays.

In some aspects, provided herein are methods of screening a sample, the methods comprising, consisting of, or consisting essentially of: (a) contacting a sample with a pooled library; (b) performing hybrid capture of nucleic acids in the sample; (c) isolating the captured nucleic acids; and (d) amplifying the isolated, captured nucleic acids. In some embodiments, the methods further comprise, consist of, or consist essentially of (e) performing high throughput sequencing analysis of the amplified nucleic acids produced in step (d). In some embodiments, the pooled library is a gene targeting library. In some embodiments, the pooled library is a reporter library for massively parallel reporter assays.

In some aspects, provided herein are methods of preparing a pooled reporter library for high throughput sequencing, the methods comprising, consisting of, or consisting essentially of: (a) performing hybrid capture of nucleic acids in a sample comprising a pooled reporter library; (b) isolating the captured nucleic acids; and (c) amplifying the isolated, captured nucleic acids. In some embodiments, the pooled reporter library comprises a promoter library, an enhancer library, or a library of regulatory elements. In some embodiments, the methods further comprise, consist of, or consist essentially of (d) performing high throughput sequencing analysis of the amplified nucleic acids produced in step (c).

In some embodiments, the pooled library comprises, consists of, or consists essentially of a nucleic acid constant region. In some embodiments, the constant region is a promoter, selectable marker, origin of replication, Cas9 gene, a viral vector backbone, a nucleic acid encoding a fluorescent protein, a nucleic acid encoding a peptide tag, or a fragment of each thereof.

In some embodiments, the pooled library is a gene targeting library or an mRNA targeting library. In some embodiments, the pooled library comprises, consists of, or consists essentially of one or more targeting nucleic acids selected from guide RNAs, shRNAs, siRNAs, and miRNAs. In some embodiments, the targeting nucleic acids are stably integrated into the genomic DNA of the sample.

In some embodiments, the pooled library is a reporter library for massively parallel reporter assays. In some embodiments, the pooled reporter library comprises, consists of, or consists essentially of one or more regulatory elements. In some embodiments, the regulatory elements are selected from promoters, enhancers, and introns. In some embodiments, the reporter elements are stably integrated into the genomic DNA of the sample.

In some embodiments, the hybrid capture of nucleic acids is performed using one or more probes that bind to a constant region in at least one targeting nucleic acid. In some embodiments, the probe comprises, consists of, or consists essentially of RNA, DNA, or LNA. In some embodiments, the probe comprises, consists of, or consists essentially of RNA. In some embodiments, the probe comprises, consists of, or consists essentially of one or more biotinylated nucleotides. In some embodiments, the probe comprises, consists of, or consists essentially of 10 to 150 nucleotides. In some embodiments, the probe comprises, consists of, or consists essentially of 20 to 200 nucleotides. In some embodiments, the probe comprises, consists of, or consists essentially of 10 to 500 nucleotides. In some embodiments, the probe comprises, consists of, or consists essentially of 20 to 1000 nucleotides. In some embodiments, the probe comprises, consists of, or consists essentially of 300 to 3000 nucleotides.

In some embodiments, the hybrid capture is performed in solution. In other embodiments, the hybrid capture is performed on a solid support. In some embodiments, the solid support is an array.

In some embodiments, the hybrid capture is performed in the presence of a buffer selected from the group of: array target hybridization buffer, saline-sodium citrate (SSC) buffer, standard hybridization buffer, formamide hybridization buffer, and Church and Gilbert's hybridization buffer. In some embodiments, the hybridization buffer comprises, consists of, or consists essentially of a buffering agent, a salt, a denaturing agent, and a chelating agent. In some embodiments, the buffering agent is selected from the group of Tris, HEPES, PIPES, PBS, MES, and MOPS. In some embodiments, the salt is selected from the group of NaCl, LiCL, KCl, and NH4Cl. In some embodiments, the denaturing agent is Urea. In some embodiments, the chelating agent is selected from the group of EDTA, citric acid, EGTA, and NTA. In some embodiments, the buffer further comprises one or more ionic detergents, non-ionic detergents, and/or reducing agents.

In some embodiments, the methods further comprise adding at least one adapter to the isolated, captured nucleic acids.

In some embodiments, provided herein are hybridization buffers for use in performing the methods, the buffers comprising, consisting of, or consisting essentially of a buffering agent, a salt, a denaturing agent, and a chelating agent, wherein the buffering agent is selected from the group of Tris, HEPES, PIPES, PBS, MES, and MOPS; wherein the salt is selected from the group of NaCl, LiCl, KCl, and NH₄Cl; wherein the denaturing agent is Urea; and wherein the chelating agent is selected from the group of EDTA, citric acid, EGTA, and NTA. In some embodiments, the buffer further comprises one or more ionic detergents, non-ionic detergents, and/or reducing agents. In some embodiments, the buffering agent is TRIS-HCl, the salt is LiCl, and the chelating agent is EDTA.

### BRIEF DESCRIPTION OF THE FIGURES

FIG. 1A and FIG. 1B: Successful library amplification. FIG. 1A depicts the guide RNAs and a ladder. FIG. 1B depicts the sample intensity of the guide RNAs. Library of guide RNA sequences is a single band observed capturing guide RNA flanking sequences from 1.8 μg of DNA followed by 18 cycles of PCR amplification.

FIG. 2A and FIG. 2B: Optimized capture and library amplification. FIG. 2A depicts the guide RNAs and a ladder. Lane AO contains a D1000 Ladder. Lane Al contains 24%-1/15/12c/3/4. Lane B1 contains 6%-1/15-12c-3/4. Lane Cl contains 1.5%-1/15-12c-3/4. Lane D1 contains KoDNA-7 cycles -1:10 dilution. FIG. 2B depicts the sample intensity of the guide RNAs. Library is a single band after capturing guide RNA flanking sequences from 13.5 μg of DNA followed by 12 cycles of PCR amplification. Values corresponding to this figure are presented in Table 2.

FIG. 3A and FIG. 3B: Model of preparation method. FIG. 3A depicts an embodiment of the first half of the method. FIG. 3B depicts an embodiment of a continuation of the method (an embodiment of the second half of the method).

FIG. 4: The required number of PCR cycles is limited by increasing input DNA. The claimed methods overcome this issue by significantly reducing the number of PCR cycles required.

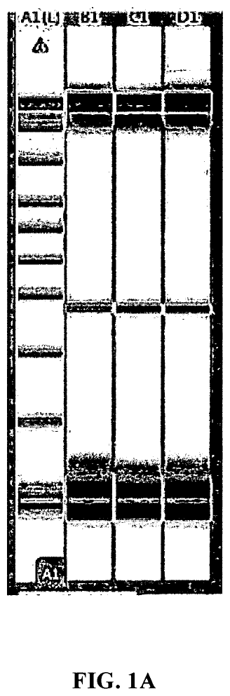

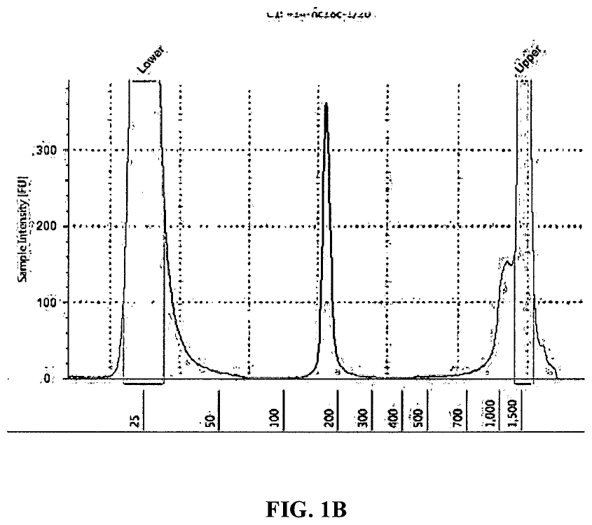

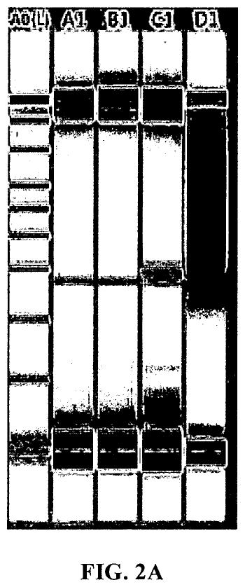

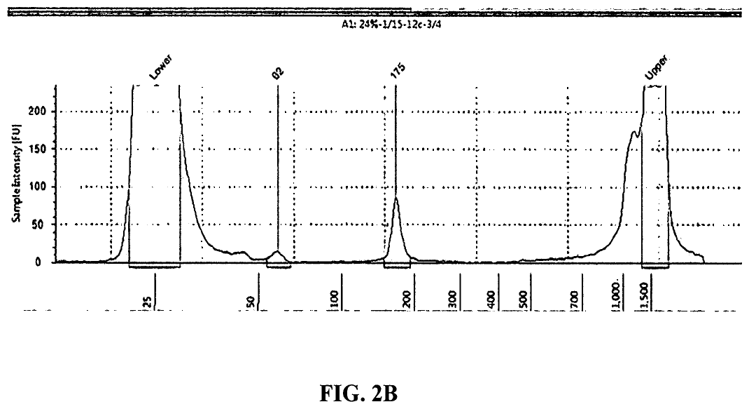

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

The practice of the present technology will employ, unless otherwise indicated, conventional techniques of tissue culture, immunology, molecular biology, microbiology, cell biology, and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

Throughout this disclosure, various publications, patents and published patent specifications are referenced by an identifying citation or by an Arabic numeral. The full citation for the publications identified by an Arabic numeral are found immediately preceding the claims. The disclosures of these publications, patents and published patent specifications are hereby incorporated by reference into the present disclosure in their entirety to more fully describe the state of the art to which this invention pertains.

Definitions

As used in the description of the invention and the appended claims, the singular forms “a,” “an” and “the” are intended to include the plural forms as well, unless the context clearly indicates otherwise.

As used herein, the term “adapter” refers to an oligonucleotide that can provide additional function or utility to a primer. For example, an adapter can encode a polymerase binding site, a restriction enzyme recognition site, or a barcode for later identification and data deconvolution.

As used herein, the term “comprising” is intended to mean that the compositions and methods include the recited elements, but do not exclude others. As used herein, the transitional phrase consisting essentially of (and grammatical variants) is to be interpreted as encompassing the recited materials or steps and those that do not materially affect the basic and novel characteristic(s) of the recited embodiment. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure.

The term “about,” as used herein when referring to a measurable value such as an amount or concentration and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

Also as used herein, the term “array” refers to a multiplex assay affixed to or immobilized on a solid support. In some embodiments, the array comprises nucleic acid targets affixed to or immobilized on a solid support. Nonlimiting examples of arrays include solid-phase arrays, bead arrays, microarrays, macroarrays, biochips, DNA chips, GeneChip® technology (Affymetrix, Inc.), DNA microarrays, gene arrays, gene expression arrays, RNA microarrays, protein arrays, tiling arrays, double-stranded B-DNA microarrays, double-stranded Z-DNA microarrays, and multi-stranded DNA microarrays. A “solid support” is a solid surface to which a multiplex assay can be affixed or immobilized. In some embodiments, the solid support comprises a planar substrate. Nonlimiting examples of solid support materials include glass, an ion selective membrane, quartz, silicon, borosilicate, and plastic.

The term “Cas9” refers to a CRISPR associated endonuclease referred to by this name. Non-limiting exemplary Cas9s include Streptococcus pyogenes Cas9 (“spCas9”), nuclease dead Cas9, and orthologs and biological equivalents each thereof. Orthologs include but are not limited to Staphylococcus aureus Cas9, (“saCas9”), Cas 9 from Streptococcus thermophiles, Legionella pneumophilia, Neisseria lactamica, Neisseria meningitides, Francisella novicida; and Cpf1 (which performs cutting functions analogous to Cas9) from various bacterial species including Acidaminococcus spp. and Francisella novicida U112.

The term “cell” as used herein may refer to either a prokaryotic or eukaryotic cell, optionally obtained from a subject or a commercially available source.

The term “constant region” as used herein refers to any nucleic acid sequence or region in a library or pooled library that does not vary between clones. For example, in a library that comprises cloning vectors, the sequence of the cloning vector backbone is constant while the sequence of the insert (e.g., a cDNA or gene) is variable. Thus, in some embodiments, a suitable constant region can comprise any non-variable sequence within a vector backbone.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human, e.g., HEK293 cells and 293 T cells.

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

As used herein, the term “CRISPR” refers to Clustered Regularly Interspaced Short Palindromic Repeats (CRISPR). CRISPR may also refer to a technique or system of sequence-specific genetic manipulation relying on the CRISPR pathway. A CRISPR recombinant expression system can be programmed to cleave a target polynucleotide using a CRISPR endonuclease and a guideRNA or a combination of a crRNA and a tracrRNA. A CRISPR system can be used to cause double stranded or single stranded breaks in a target polynucleotide such as DNA or RNA. A CRISPR system can also be used to recruit proteins or label a target polynucleotide. In some aspects, CRISPR-mediated gene editing utilizes the pathways of nonhomologous end-joining (NHEJ) or homologous recombination to perform the edits. These applications of CRISPR technology are known and widely practiced in the art. See, e.g., U.S. Pat. No. 8,697,359 and Hsu et al. (2014) Cell 156(6): 1262-1278.

The term “gRNA” or “guide RNA” as used herein refers to the guide RNA sequences used to target specific genes for correction employing the CRISPR technique. Techniques of designing gRNAs and donor therapeutic polynucleotides for target specificity are well known in the art. For example, Doench, J., et al. Nature biotechnology 2014; 32(12):1262-7, Mohr, S. et al. (2016) FEBS Journal 283: 3232-38, and Graham, D., et al. Genome Biol. 2015; 16: 260, each incorporated herein in their entirety. gRNA comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some embodiments, a gRNA is synthetic (Kelley, M. et al. (2016) J of Biotechnology 233 (2016) 74-83, incorporated by reference herein in its entirety). In some embodiments, a gRNA is engineered to have one or more modifications that improve specificity, binding, or other features of the gRNA. In some embodiments, a gRNA is an enhanced gRNA (“esgRNA”) (Chen B, et al. Cell. 2013;155:1479-1491. doi: 10.1016/j.ce11.2013.12.001, incorporated by reference herein in its entirety).

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

The terms “equivalent” or “biological equivalent” are used interchangeably when referring to a particular molecule, biological, or cellular material and intend those having minimal homology while still maintaining desired structure or functionality. Non-limiting examples of equivalent polypeptides, include a polypeptide having at least 60%, or alternatively at least 65%, or alternatively at least 70%, or alternatively at least 75%, or alternatively 80%, or alternatively at least 85%, or alternatively at least 90%, or alternatively at least 95% identity thereto or for polypeptide sequences, or a polypeptide which is encoded by a polynucleotide or its complement that hybridizes under conditions of high stringency to a polynucleotide encoding such polypeptide sequences. Conditions of high stringency are described herein and incorporated herein by reference. Alternatively, an equivalent thereof is a polypeptide encoded by a polynucleotide or a complement thereto, having at least 70%, or alternatively at least 75%, or alternatively 80%, or alternatively at least 85%, or alternatively at least 90%, or alternatively at least 95% identity, or at least 97% sequence identity to the reference polynucleotide, e.g., the wild-type polynucleotide.

Non-limiting examples of equivalent polypeptides, include a polynucleotide having at least 60%, or alternatively at least 65%, or alternatively at least 70%, or alternatively at least 75%, or alternatively 80%, or alternatively at least 85%, or alternatively at least 90%, or alternatively at least 95%, or alternatively at least 97%, identity to a reference polynucleotide. An equivalent also intends a polynucleotide or its complement that hybridizes under conditions of high stringency to a reference polynucleotide.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. In certain embodiments, default parameters are used for alignment. A non-limiting exemplary alignment program is BLAST, using default parameters. In particular, exemplary programs include BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. Sequence identity and percent identity can determined by incorporating them into clustalW (available at the web address:genome.jp/tools/clustalw/, last accessed on Jan. 13, 2017).

“Homology” or “identity” or “similarity” refers to sequence similarity between two peptides or between two nucleic acid molecules. Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

“Homology” or “identity” or “similarity” can also refer to two nucleic acid molecules that hybridize under stringent conditions.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5 ×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

As used herein, “expression” refers to the process by which polynucleotides are transcribed into an RNA and/or the process by which the transcribed RNA is subsequently translated into peptides, polypeptides, or proteins. If the polynucleotide is derived from genomic DNA, expression may include splicing of the mRNA in an eukaryotic cell.

The term “isolated” as used herein refers to molecules or biologicals or cellular materials being substantially free from other materials. In one aspect, the term “isolated” refers to nucleic acid, such as DNA or RNA, or protein' or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized.

As used herein, the term “functional” may be used to modify any molecule, biological, or cellular material to intend that it accomplishes a particular, specified effect. As used herein, “loss-of-function” refers to an effect that reduces or eliminates the normal activity of a molecule.

As used herein, the terms “nucleic acid sequence,” “oligonucleotide,” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

As used herein, the term “hybrid capture” refers to a quantitative nucleic acid test that uses an efficient signal amplification strategy. Methods of performing hybrid capture are known in the art and described herein, for example, in Duncavage et al. (2011) J. Mol. Diagn. 13(3): 325-33 (performs hybrid-capture target enrichment using PCR-generated capture probes);

The term “inhibitory RNA” refers to an RNA molecule capable of RNA interference, a mechanism whereby an inhibitory RNA molecule targets a messenger RNA (mRNA) molecule, resulting in inhibition gene expression and/or translation. RNA interference is also known as post-transcriptional gene silencing. Exemplary inhibitory RNAs include but are not limited to antisense RNAs, microRNAs (miRNA), small interfering RNAs (siRNA), short hairpin RNAs (shRNA), double stranded RNA (dsRNA) and intermediates thereof. Methods of designing, cloning, and expressing inhibitory RNAs are known in the art (e.g. McIntyre et al, BMC Biotechnol. 2006; 6:1; Moore et al. Methods Mol. Biol. 2010; 629: 141-158) and custom RNAi kits are commercially available (e.g. GeneAssist™ Custom siRNA Builder, ThermoFisher Scientific, Waltham, Mass.).

As used herein, “minimal” refers to the elements of a functional sequence that are necessary to allow function of the sequence. For example, a minimal promoter comprises a TATA box and transcription initiation site.

As used herein, “pooled library” refers to a collection of nucleic acids that is stored and propagated in a pooled population. In some embodiments, a pooled library comprises a preparation of different plasmids or other nucleic acids for use in a screen. In some embodiments, the pooled library is a gene targeting library or an mRNA targeting library. In some embodiments, the pooled library is a CRISPR-based targeting library. In some embodiments, the pooled library is a shRNA library for screening or targeting. In some embodiments, the pooled library is a reporter library. Nonlimiting examples of reporter libraries include massively parallel reporter assay libraries such as libraries for splicing regulatory elements (e.g., Soemedi, R. et al. Nature Genetics volume 49, pages 848-855 (2017), incorporated by reference herein in its entirety) and libraries for enhancer and/or promoter regulatory elements (e.g., Patwardhan, R. et al. Nature Biotechnology volume 30, pages 265-270 (2012), incorporated by reference herein in its entirety).

Plasmids within a given pooled library have the same vector backbone but they each express, target, or comprise different inserts. In some embodiments, an insert comprises all or part of a gene, cDNA, shRNA, RNAi, miRNA, guide RNA, barcode, expression control element, and/or a random nucleic acid sequence. For example, in a cDNA library, each plasmid contains a unique cDNA insert. In shRNA or gRNA libraries, each plasmid contains a unique gene targeting sequence insert (but there may be multiple sequences targeting each gene in the overall library). Barcoding libraries contain plasmids with unique, semi-random sequence inserts that can be used for applications like lineage tracing or parsing the effects of expressing multiple genes at once. Pooled libraries can be small if designed to cover only a subset of genes or targets, or very large. For example, the Toronto KnockOut library has over 175,000 different gRNA-containing plasmids. Pooled libraries represent a powerful tool for forward genetic screening and identifying previously unknown genes that contribute to a phenotype.

The term “regulatory element” is used interchangeably with “expression control element” and is used herein to refer to any nucleic acid sequence that regulates the expression and/or splicing of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. An “enhancer” is a region of DNA that can be bound by activating proteins to increase the likelihood or frequency of transcription. In some embodiments, the regulatory element is a promoter or enhancer.

The term “sample” as used herein relates to a material or mixture of materials, typically, although not necessarily, in liquid form, containing one or more analytes of interest. The nucleic acid samples used herein may be complex in that they contain multiple different molecules that contain sequences. Fragmented genomic DNA and cDNA made from mRNA from a mammal (e.g., mouse or human) are types of complex samples. Complex samples may have more then 10⁴, 10⁵, 10⁶ or 10⁷ different nucleic acid molecules. A DNA target may originate from any source such as genomic DNA, cDNA (from RNA) or artificial DNA constructs. Any sample containing nucleic acid, e.g., genomic DNA made from tissue culture cells, a sample of tissue, or an FPET samples, may be employed herein. In some embodiments, the sample may comprise a library.

As used herein, the term “stably integrated” refers to a polynucleotide that is incorporated into a locus in the genome of a cell or organism, and this incorporation is durable (i.e. the polynucleotide remains integrated in the genomic locus throughout the cell cycle including through DNA replication and mitosis).

The term “target polynucleotide,” as used herein, refers to a polynucleotide of interest under study. In certain embodiments, a target polynucleotide contains one or more sequences that are of interest and under study.

### MODES OF CARRYING OUT THE DISCLOSURE

### Hybrid Capture Methods

In some aspects, provided herein are methods of preparing a pooled library for high throughput sequencing, the methods comprising, consisting of, or consisting essentially of: (a) performing hybrid capture of nucleic acids in a sample comprising a pooled library; (b) isolating the captured nucleic acids; and (c) amplifying the isolated, captured nucleic acids. In some embodiments, the methods further comprise, consist of, or consist essentially of (d) performing high throughput sequencing analysis of the amplified nucleic acids produced in step (c).

In some aspects, provided herein are methods of screening a sample, the methods comprising, consisting of, or consisting essentially of: (a) contacting a sample with a pooled library; (b) performing hybrid capture of nucleic acids in the sample; (c) isolating the captured nucleic acids; and (d) amplifying the isolated, captured nucleic acids. In some embodiments, the methods further comprise, consist of, or consist essentially of (e) performing high throughput sequencing analysis of the amplified nucleic acids produced in step (d).

In some aspects, provided herein are methods of preparing a pooled reporter library for high throughput sequencing, the methods comprising, consisting of, or consisting essentially of: (a) performing hybrid capture of nucleic acids in a sample comprising a pooled reporter library; (b) isolating the captured nucleic acids; and (c) amplifying the isolated, captured nucleic acids. In some embodiments, the pooled reporter library comprises a promoter library, an enhancer library, or a library of regulatory elements. In some embodiments, the methods further comprise, consist of, or consist essentially of (d) performing high throughput sequencing analysis of the amplified nucleic acids produced in step (c).

In some embodiments, the hybrid capture is performed in solution. Generally, solution-based target enrichment systems comprise a pool of labeled (e.g., biotinylated) oligonucleotide probes targeting the constant regions or desired genes, exons, and/or other targets of interest. These probes are then added to adapter-ligated DNA in solution for hybridization with targeted regions of interest. The hybridized probes are then captured and purified by beads (e.g., magnetic beads) and subsequently amplified and sequenced.

In some embodiments, beads suitable for use in the hybridization capture methods are magnetic. Nonlimiting examples of suitable beads include New England Biolab's Streptavidin Magnetic Beads, Catalog number: S1420S or NEB's Hydrophilic Magnetic Beads, Catalog number: S1421S, Pierce™ Streptavidin Magnetic Beads, Catalog number: 88816 or 88817, ThermoFisher Dynabeads™ MyOne™ Streptavidin T1 (catalog numbers: 65601, 65602), Dynabeads® MyOne™ Streptavidin Cl (catalog numbers: 65001, 65002), Dynabeads™ M-280 Streptavidin (catalog numbers: 60210, 11205D, 11206D), MagnaLink™ Streptavidin Magnetic Beads 2.8 pm (catalog number M-1003), NanoLink™ Streptavidin Magnetic Beads 1.0 pm (catalog number M-1002).

For example, in one embodiment, a liquid-based array is used: bead arrays are commercially available and in this embodiment, carboxylated polystyrene bead arrays are preferable. Each well of a 96-well plate, for example, has a mixture of bead sets. A 13-plex has 13 bead sets where each bead set has a specific “signature” and the signature is provided by dyes that are inside each bead. The ratio of these dyes is specific for each bead set, and enables differentiation between each of the bead sets. Capture sequence probes or oligonucleotides specific for one target nucleic acid are applied or conjugated to one particular bead set. When the target is hybridized to the bead conjugated probes or oligonucleotides, selection of a particular bead set and then detection occurs using the complementary nucleic acid probe and labeled DNA:RNA hybrid-specific binding agent. The selection or separation may be carried out in a flow-cytometer, where the beads proceed one-by-one through two lasers: one of which selects the signature on the bead, while the other detects the target as identified by the labeled DNA:RNA hybrid-specific binding agents. In this way, multiple targets may be differentiated and detected. Additionally, the labeled DNA:RNA reagent allows enhanced signal detection, thereby increasing both the specificity and sensitivity of the assay.

In other embodiments, the hybrid capture is performed on a solid support. Examples of appropriate solid supports include beads (e.g. silica gel, controlled pore glass, magnetic, Sephadex/Sepharose, cellulose), flat or planar surfaces or chips (e.g. glass fiber filters, glass surfaces, metal surface (steel, gold, silver, aluminum, copper and silicon), capillaries, plastic (e.g. polyethylene, polypropylene, polyamide, polyvinylidenedifluoride membranes or microtiter plates)); or pins or combs made from similar materials comprising beads or flat surfaces or beads placed into pits in flat surfaces such as wafers (e.g. silicon wafers). The detection of the RNA:DNA hybrid complex bound to a solid support may be performed in a multiplex format using, for example, a PE-labeled antibody, carboxylated distinguishable beads, and detected by flow-cytometry.

In some embodiments, the solid support is an array. Generally, an array-based hybrid capture is performed by first shearing the sample nucleic acid (e.g., genomic DNA) into randomly sized fragments. Sequencer-specific adapters can then be added via a PCR reaction. An immobilized probe can then be used to capture the targets in the fragmented library. Nonspecific hybrids can be washed away followed by elution of the hybridized probes.

In some embodiments, hybrid capture is performed to enrich for integrated DNA. An example of hybrid capture is provided herein. First, primer-specific amplification of genomic targets is performed to generate amplicons that can be used as bait for the capture. The amplicons are used as a template in a second PCR further incorporating a label such as biotin-14-dCTP. Genomic DNA is prepared from each of the samples to be sequenced, sheared to an average fragment size of about 50 to 1000 base pairs, 100-500 base pairs, 100-200 base pairs, 200-300 base pairs, 300-400 base pairs, or 400 to 500 base pairs. These fragments are enzymatically repaired to blunt the ends, and ligated to adapter sequences (e.g. adapter sequences suitable for next generation sequencing). About 100 ng to 1 μg, or about 250 ng to about 750 ng, or about 500 ng of genomic DNA library is denatured. The denatured library is combined with about 10 ng to about 1 μg, or about 100 to about 500 ng, or about 100 ng of the bait fragments and hybridized for 48 hours. Mixing this hybridization reaction with beads (e.g. streptavidin- or avidin-coated superparamagnetic or polymer beads) allows binding of biotinylated bait—target hybrids. These hybrids can then be selectively removed from solution by applying a magnetic field or through centrifugation, filtration, or washing. Any remaining supernatant is removed, and the beads are washed, removing nonspecific DNA or RNA. Enriched target sequences are released from the bead-bound bait sequences by basic denaturation (e.g. in 0.125 N NaOH), neutralized, and then amplified by PCR to generate double-stranded libraries that can be sequenced.

In some embodiments, the steps of isolating and amplifying the isolated captured nucleic acids are performed concurrently. In some embodiments, the hybridization of a target and probe may occur simultaneously with the capture step by a hybrid-binding agent while in the same mixture and at an elevated temperature. The elevated temperature during the entire process may allow an increase in specificity of target capture, while decreasing the reaction time. It is to be understood that the low, moderate and high stringency hybridization/washing conditions may be varied using a variety of ingredients, buffers and temperatures well known to and practiced by the skilled artisan. For additional stringency conditions, see T. Maniatis et al., Molecular Cloning: A Laboratory Manual, Cold Spring Harbor Laboratory, Cold Spring Harbor, N.Y. (1982). The one step hybridization and capture may also be more efficient than performing hybridization and capture sequentially, depending on the overall assay conditions.

In some embodiments, the methods further comprise adding at least one adapter to the isolated, captured nucleic acids. Nonlimiting examples of adapters include polymerase binding sites, restriction enzyme recognition sites, and barcodes for later identification and data deconvolution.

In some embodiments, the isolated, captured nucleic acids are identified by a method comprising or consisting of, nucleic acid sequencing, DNA sequencing, RNA sequencing, high throughput sequencing, Next Generation Sequencing (NGS) pyrosequencing, sequencing by synthesis, Ion Torrent and/or Ion proton sequencing, shotgun sequencing, and/or Sanger sequencing. Methods of performing sequencing of captured nucleic acids are described, for example, in Duncavage, E. et al. J. Mol. Diagn. 2011 May; 13(3): 325-333, incorporated herein by reference in its entirety.

The efficiency of gene targeting using the screening libraries can be assayed by any method known in the art, including by PCR validation of the targeted allele, and/or by utilizing reporter loci and quantitating the amount of gene targeting that has been successfully completed.

### Constant Regions and Libraries

By specifically capturing the desired constant region (both strands of integrated vector are captured) out of the 3 billion base genome, the claimed methods wash away the unrelated DNA that drives amplification issues and creates libraries that are: highly correlated across biological replicates and capture true signal with less processing and sequencing.

In some embodiments, the pooled library comprises a nucleic acid constant region. In some embodiments, the constant region is a promoter, intron, enhancer, selectable marker, origin of replication, Cas9 gene, a viral vector backbone, a reporter gene such as a nucleic acid encoding a fluorescent protein, a nucleic acid encoding a peptide tag, a minimal promoter region, a minimal enhancer region, a minimal splice site region, a minimal 5′ or 3′ untranslated region, or a fragment of each thereof. In some embodiments, the constant region is a uniform sequence tag or barcode that has been added to each member of the library. In some embodiments, the constant region comprises, consists of, or consists essentially of all or part of a vector, viral genome, or plasmid. In some embodiments, the constant region comprises, consists of, or consists essentially of all or part of a viral vector backbone such as a lentivirus, adenovirus, or adeno-associated virus (AAV).

In some embodiments, the constant region comprises, consists of, or consists essentially of 10 to 150 nucleotides. In some embodiments, the constant region comprises, consists of, or consists essentially of 20 to 200 nucleotides. In some embodiments, the constant region comprises, consists of, or consists essentially of 10 to 500 nucleotides. In some embodiments, the constant region comprises, consists of, or consists essentially of 20 to 1000 nucleotides. In some embodiments, the constant region comprises, consists of, or consists essentially of 20 to 10,000 nucleotides. In some embodiments, the constant region comprises, consists of, or consists essentially of about 10, about 20, about 30, about 40, about 50, about 60, about 70, about 80, about 90, or about 100 nucleotides. In some embodiments, the constant region comprises, consists of, or consists essentially of about 20 to about 10,000 nucleotides. In some embodiments, the constant region comprises, consists of, or consists essentially of all or part of a vector, viral genome, or plasmid up to 27,000 nucleotides in length.

In some embodiments, the pooled library is a gene targeting library or an mRNA targeting library. In some embodiments, the pooled library comprises, consists of, or consists essentially of one or more targeting nucleic acids selected from guide RNAs, shRNAs, siRNAs, and miRNAs. In some embodiments, the targeting nucleic acids are stably integrated into the genomic DNA of the sample.

In some embodiments, the pooled library is a reporter library for massively parallel reporter assays. In some embodiments, the pooled reporter library comprises, consists of, or consists essentially of one or more regulatory elements. In some embodiments, the regulatory elements are selected from promoters, enhancers, and introns. In some embodiments, the reporter elements are stably integrated into the genomic DNA of the sample.

In some embodiments, the library is a genome-scale CRISPR-Cas knockout library that utilizes lentiviral delivery of a genome-scale CRISPR-Cas9 knockout library targeting all or a subset of the genes of an organism with unique guide sequences. In some aspects, the screening library is an RNAi library comprising shRNAs, siRNAs, or miRNAs designed to target all or a subset of the genes of an organism.

### Probes

In some embodiments, the hybrid capture of nucleic acids is performed using one or more nucleic acid probes. Nucleic acid probes are detectable nucleic acid sequences that hybridize to complementary RNA or DNA sequences in a test sample. Detection of the probe indicates the presence of a particular nucleic acid sequence in the test sample. In some embodiments, the probe binds to all or part of a constant region in at least one targeting nucleic acid.

In some embodiments, the probe comprises, consists of, or consists essentially of 10 to 150 nucleotides. In some embodiments, the probe comprises, consists of, or consists essentially of 20 to 200 nucleotides. In some embodiments, the probe comprises, consists of, or consists essentially of 10 to 500 nucleotides. In some embodiments, the probe comprises, consists of, or consists essentially of 20 to 1000 nucleotides. In some embodiments, the probe comprises, consists of, or consists essentially of 300 to 3000 nucleotides. In some embodiments, the probe comprises, consists of, or consists essentially of about 10, about 20, about 30, about 40, about 50, about 60, about 70, about 80, about 90, or about 100 nucleotides. In a preferred embodiment, the length of the probe is between 50-1000 nucleotides. In some embodiments, the length of the probe is up to 10%, 20%, 30%, 40%, 50%, 60%, 70%, 80%, 90%, or 100% of the target nucleic acid. In some embodiments, the probes specifically hybridize to the target nucleic acid under conditions of high or moderate stringency. In some embodiments, the target nucleic acid comprises a constant region.

The sequence of a probe is preferably at least 75%, at least 80%, at least 85%, at least 90%, at least 95%, at least 96%, at least 97%, at least 98%, at least 99%, or 100% complementary to the target hybridization region (e.g., constant region). In some embodiments, the probe is 100% complementary to this sequence. In some embodiments, the probe contains less than 75%, less than 50%, less than 25%, or less than 10% sequence identity to non-desired sequences believed to be present in a test sample.

In some embodiments, the sequence within a target nucleic acid to which a probe binds (e.g., constant region) is about 5, about 10, about 15, about 20, about 25, about 30, about 35, about 40, about 50, about 60, about 70, about 80, about 90, about 100, about 125, about 150, about 175, about 200, about 300, about 400, about 500, about 600, about 700, about 800, about 900, or about 1000 nucleotides in length. In particular embodiments, the sequence within the target nucleic acid to which the probe bines is about 20 to about 40 nucleotides in length. In some embodiments, the sequences to which the probe hybridizes are unique sequences or group-specific sequences. Group-specific sequences are multiple related sequences that form discrete groups.

In some embodiments, the probe comprises, consists of, or consists essentially of DNA, RNA, peptide nucleic acids (PNAs), locked nucleic acids (LNAs), or other nucleic acid analogues. A “locked nucleic acid” as defined herein is a novel class of oligonucleotide analogues which form duplexes with complementary DNA and RNA with high thermal stability and selectivity. The usual conformational freedom of the furanose ring in standard nucleosides is restricted in LNAs due to the methylene linker connecting the 2′-O position to the 4′-C position. PNAs are oligonucleotides in which the sugar-phosphate backbone is replaced with a polyamide or “pseudopeptide” backbone. In some embodiments, the probe is comprises, consists of, or consists essentially of DNA. In some embodiments, the probe comprises, consists of, or consists essentially of single stranded DNA. In some embodiments, the probe comprises, consists of, or consists essentially of RNA. In some embodiments, the probe comprises, consists of, or consists essentially of one or more synthetic nucleotides. In some embodiments, the probe is synthetic.

In some embodiments, the probe is detectably labeled. In some embodiments, the label is a fluorescent, chemiluminescent, radioactive, or magnetic label. In some embodiments, the label is biotin. In some embodiments, the probe comprises one or more biotinylated nucleotides. Non-limiting examples of biotinylated nucleotides include: bio-11-UTP, bio-16-UTP, bio-14-CTP, bio-16-CTP, etc).

In some embodiments, the probe contains one or more modifications in the nucleic acid which allows specific capture of the probe onto a solid phase. For example, the probe can be modified by tagging it with at least one ligand by methods well-known to those skilled in the art including, for example, nick-translation, chemical or photochemical incorporation. In addition, the probe may be tagged at multiple positions with one or multiple types of labels. For example, the probe may be tagged with biotin, which binds to streptavidin; or digoxigenin, which binds to anti-digoxigenin; or 2,4-dinitrophenol (DNP), which binds to anti-DNP. Fluorogens can also be used to modify the probes. Examples of fluorogens include fluorescein and derivatives, phycoerytlrin, allo-phycocyanin, phycocyanin, rhodamine, Texas Red or other proprietary fluorogens. The fluorogens are generally attached by chemical modification and bind to a fluorogen-specific antibody, such as anti-fluorescein. It will be understood by those skilled in the art that the probe can also be tagged by incorporation of a modified base containing any chemical group recognizable by specific antibodies. Other tags and methods of tagging nucleotide sequences for capture onto a solid phase coated with substrate are well known to those skilled in the art. A review of nucleic acid labels can be found in the article by Landegren, et al, “DNA Diagnostics-Molecular Techniques and Automation”, Science, 242:229-237 (1988), which is incorporated herein by reference. In one preferred embodiment, the probe is tagged with biotin on both the 5′ and the 3′ ends of the nucleotide sequence. In another embodiment, the probe is not modified but is captured on a solid matrix by virtue of sequences contained in the probe capable of hybridization to the matrix.

The probes can be produced by any suitable method known in the art, including for example, by chemical synthesis, isolation from a naturally-occurring source, recombinant production and asymmetric PCR (McCabe, 1990 In: PCR Protocols: A guide to methods and applications. San Diego, Calif., Academic Press, 76-83, incorporated herein by reference). It may be preferred to chemically synthesize the probes in one or more segments and subsequently link the segments. Several chemical synthesis methods are described by Narang et al. (1979 Meth. Enzynol. 68:90), Brown et al. (1979 Meth. Enzymol. 68:109) and Caruthers et al. (1985 Meth. Enzymol. 154:287), each of which are incorporated herein by reference. Alternatively, cloning methods may provide a convenient nucleic acid fragment which can be isolated for use as a promoter primer. A double-stranded DNA probe can be rendered single-stranded using, for example, conventional denaturation methods prior to hybridization to the target nucleic acids.

### Hybridization Buffers

In some embodiments, the hybrid capture is performed in the presence of a buffer selected from the group of: array target hybridization buffer, saline—sodium citrate (SSC) buffer, standard hybridization buffer, formamide hybridization buffer, and Church and Gilbert's hybridization buffer. In some embodiments, the hybridization buffer comprises, consists of, or consists essentially of a buffering agent, a salt, a denaturing agent, and a chelating agent. In some embodiments, the buffering agent is selected from the group of TRIS, TRIS-HCI, HEPES, PIPES, PBS, MES, and MOPS. In some embodiments, the salt is selected from the group of NaCl, LiCL, KCl, and NH4Cl. In some embodiments, the denaturing agent is Urea. In some embodiments, the chelating agent is selected from the group of EDTA, citric acid, EGTA, and NTA. In some embodiments, the buffer further comprises one or more ionic detergents, non-ionic detergents, and/or reducing agents.

In some embodiments, the hybrid capture buffer is as described in Solution Hybrid Selection with Ultra-long Oligonucleotides for Massively Parallel Targeted Sequencing (Nat Biotechnol. 2009 February;27(2):182-9. doi: 10.1038/nbt.1523, incorporated herein by reference in its entirety); 2× hybridization buffer (10× SSPE, 10× Denhardt's, 10 mM EDTA and 0.2% SDS), Array Target Hybridization Buffer (Final 1× concentration is 100 mM MES, 1M [Na+], 20 mM EDTA, 0.01% Tween-20) 50 mL, 8.3 mL of 12× MES Stock Buffer, 17.7 mL of 5M NaCl, 4.0 mL of 0.5M EDTA, 0.1 mL of 10% Tween-20, 19.9 mL of water (https://openwetware.org/wiki/Affymetrix_Target_Hybridization, incorporated herein by reference in its entirety, saline-sodium citrate (SSC) buffer (a 20× stock solution consists of 3 M sodium chloride and 300 mM trisodium citrate (adjusted to pH 7.0 with HCl), Standard Hybridization Buffer (5× SSC 0.1% (w/v) N-lauroylsarcosine 0.02% (w/v) SDS 1% Blocking Reagent, (http://www.img.bio.uni-goettingen.de/ms-www/internal/methods/DNA/Roche_Dig/023.pdf, incorporated herein by reference in its entirety), Formamide hybridization buffer (50% Formanide, 2 SSC, 10% dextran sulfate (pH 7)), and Church And Gilbert's Hybridization Buffer: (1mM EDTA (ethylenediaminetetracetic acid), 1% BSA (bovine serum albumin), 0.5M NaH2PO4 (sodium phosphate, monobasic) 7% SDS (sodium dodecyl sulfate, adjusted to pH 7.2).

TABLE 1
Exemplary Hybridization Buffer

| Reagent | Final Concentration | Optimal range | Suitable Alternative Reagent |
|---|---|---|---|
| Buffering agent: | 25 mM | Concentration: 10-100 mM | Any buffering compound which |
| Tris-HCL, pH 7.4 | | pH-6.5-8.5 | will have “save” for RNA and |
| | | | DNA pH and concertation at |
| | | | temperatures, for RNA: 25° C.- |
| | | | 70° C., for DNA 25° C.-95° C. |
| | | | Some examples of buffering |
| | | | agents are: |
| | | | Tris, HEPES, PIPES, PBS, MES, |
| | | | MOPS, and many others¹. |
| Salt: | 400 mM | 50-1000 mM | Most monovalent salts, which is |
| LiCl | | | “save” for RNA. Mostly used: |
| | | | NaCl, LiCL, KCl, NH4Cl. |
| Urea, denaturing | 1M | 0.5M-8M | |
| agent | | | |
| Chelating agent: | 5 mM | 0.1-50 mM | Citric acid, EDTA, EGTA, NTA |
| EDTA | | | and many others - EDTA will |
| | | | “protect RNA” from degrading |
| | | | divalent metals (Mg, Ca, etc) |
| Optional: | 0.1% | 0.01%-1% | Most non-ionic detergents, mostly |
| detergent NP-40 | | | to keep beads non-aggregated |
| Optional: | 0.1% | 0.01%-1% | Most ionic detergents, mostly to |
| detergent Sodium | | | keep beads non-aggregated |
| deoxycholate | | | |
| Optional: | 0.1% | 0.01%-1% | Most ionic detergents, mostly to |
| detergent SDS | | | keep beads non-aggregated and to |
| | | | inactivate RNAsel bacterial |
| | | | RNAse. |
| Optional: | 10 mM | 1-100 mM | TCEP, DTT, B2M - protect RNA |
| reducing agents, | | | from degradation. |
| like DTT | | | |

### Kits

In some aspects, provided herein are kits comprising, consisting of, or consisting essentially of one or more reagents useful for performing the methods described herein. Non-limiting examples of such reagents include one or more probes, labeled probes, pooled libraries (e.g., gene targeting libraries, nucleic acid libraries, and screening libraries), transfection reagents, transduction reagents, hybridization buffer, and PCR primers. In some embodiments, the kits comprise, consist of, or consist essentially of one or more probes specific for a constant region and a hybridization buffer. In some embodiments, the kits further comprise, consist of, or consist essentially of instructions for use. In some embodiments, the hybridization buffer is provided at a 2×, 3×, 4×, 5×, 10×, 15×, 20×, 40×, 50×, or 100× concentration.

### EXAMPLE 1

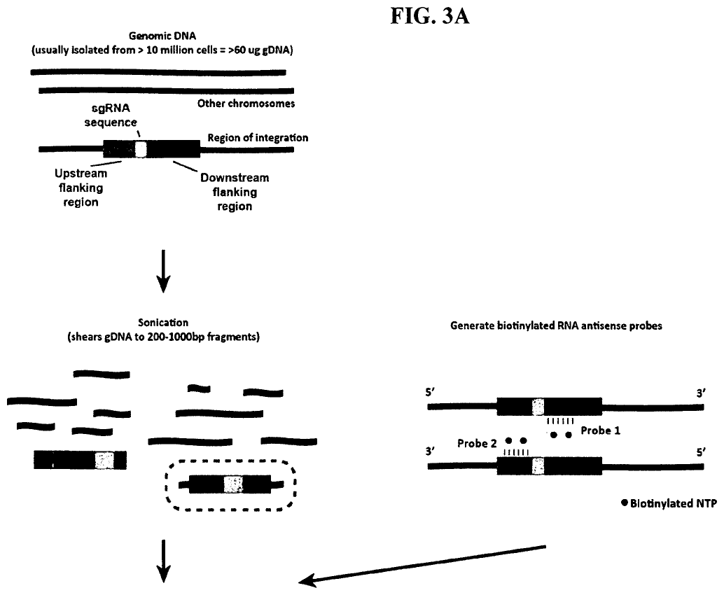

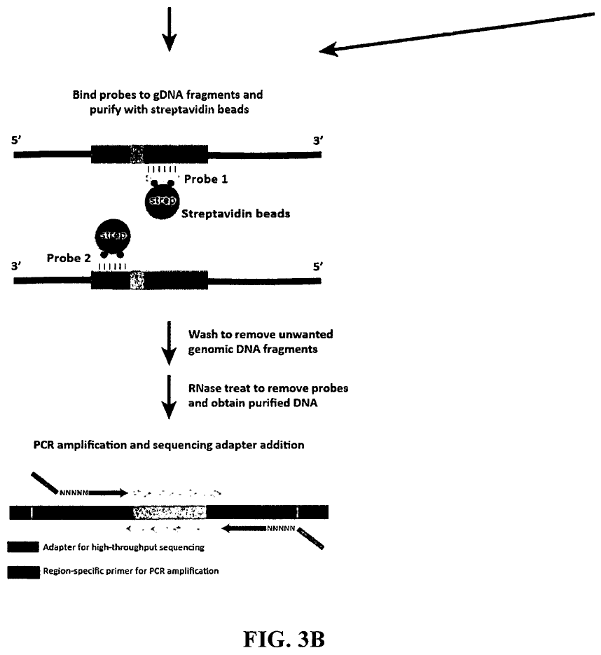

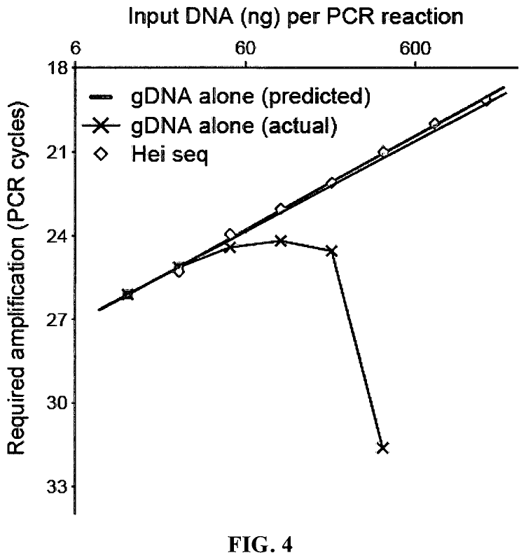

An overview of exemplary embodiments of the methods are provided in FIGS. 3A-3B. After integration of the sgRNA sequence, the sample is sonicated to shear the gDNA into 200-1000 bp fragments. Biotinylated RNA antisense probes are generated to the flanking regions using biotinylated NTP (FIG. 3A). Next, the probes are bound to gDNA fragments and purified with streptavidin beads. The samples are washed to remove unwanted genomic DNA fragments and then RNase treated to remove the probes and obtain purified DNA. Finally, PCR amplification is performed with region-specific primers attached to adapters for high throughput sequencing (FIG. 3B).

After genomic DNA isolation and fragmentation, hybrid capture is performed using biotinylated antisense RNA probes specifically recognizing constant regions in the integrated DNA fragment that flank the variable genetargeting region (containing sgRNA sequence). After hybridization, these regions are isolated by binding biotinylated RNA probes (and bound DNA) to streptavidin beads, followed by washing. DNA is then isolated by RNase digestion and degradation of RNA probes and standard DNA extraction. PCR is then used to amplify the variable genetargeting region (containing sgRNA sequence) and to add adapters for high throughput sequencing.

Additional steps of the library preparation methods are described, for example, in Shalem et al. (2014) Science 343(6166): 84-87, incorporated herein by reference in its entirety.

### EXAMPLE 2

The CRISPR library used for this example is the GeCKO library, described in Shalem et al. (2014) Science 343(6166): 84-87, incorporated herein by reference in its entirety. Hybrid capture was performed in solution, as described in Gnirke, A. et al. (2009) Nat. Biotechnol. 27(2): 182-9, incorporated herein by reference in its entirety.

TABLE 2

| Size | Concentration | Peak Molarity | |
|---|---|---|---|
| (base pairs) | [ng/μL] | [nmol/l] | Observations |
| 25 | 6.61 | 407 | Lower Marker |
| 62 | 0.0291 | 0.725 | |
| 175 | 0.143 | 1.26 | |
| 1500 | (6.50) | 6.67 | Upper Marker |

Applicant has implemented the full hybrid capture protocol on test samples, and observed greater than 70% capture efficiency in capturing sgRNA sequence out of total genomic DNA, with greater than 1,000 fold enrichment of sgRNA sequences in purified sample relative to supernatant. Validation experiments will show increased robustness across technical and biological replicate experiments.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

1. A method of preparing a pooled gene targeting library for high throughput sequencing, the method comprising:
(a) performing hybrid capture of nucleic acids in a sample comprising a pooled library;
(b) isolating the captured nucleic acids; and
(c) amplifying the isolated, captured nucleic acids.

2. The method of claim 1, wherein the pooled gene targeting library comprises a nucleic acid constant region.

3. The method of claim 2, wherein the constant region is a promoter, selectable marker, origin of replication, Cas9 gene, a viral vector backbone, a nucleic acid encoding a fluorescent protein, a nucleic acid encoding a peptide tag, a minimal promoter region, a minimal enhancer region, a minimal splice site region, a minimal 5′ or 3′ untranslated region, or a fragment of each thereof.

4. The method of claim 1, further comprising (d) performing high throughput sequencing analysis of the amplified nucleic acids produced in step (c).

5. The method of claim 1, wherein the pooled library comprises one or more targeting nucleic acids selected from guide RNAs, shRNAs, siRNAs, and miRNAs.

6. The method claim 3, wherein the targeting nucleic acids are stably integrated into the genomic DNA of the sample.

7. The method of claim 2, wherein the hybrid capture of nucleic acids is performed using one or more probes that bind to the constant region.

8-9. (canceled)

10. The method of claim 5, wherein:
the probe comprises RNA, DNA, or LNA; or
the probe comprises one or more biotinylated nucleotides.

11-14. (canceled)

15. The method of claim 5, wherein the probe comprises 10 to 3000 nucleotides.

16. (canceled)

17. The method of claim 1, wherein the hybrid capture is performed in a solution or on a solid support.

18. The method of claim 17, wherein the solid support is an array.

19. The method of claim 1, wherein the hybrid capture is performed in the presence of a buffer selected from the group of: array target hybridization buffer, saline—sodium citrate (SSC) buffer, standard hybridization buffer, formamide hybridization buffer, and Church and Gilbert's hybridization buffer.

20. The method of claim 1, wherein the hybridization buffer comprises a buffering agent, a salt, a denaturing agent, and a chelating agent.

21. The method of claim 20, wherein
the buffering agent is selected from the group of TRIS, HEPES, PIPES, PBS, MES, and MOPS;
wherein the salt is selected from the group of NaCl, LiCl, KCl, and NH4Cl;
wherein the denaturing agent is Urea;
wherein the chelating agent is selected from the group of EDTA, citric acid, EGTA, and NTA: or
wherein the hybridization buffer further comprises one or more ionic detergents, non-ionic detergents, and/or reducing agents.

22-25. (canceled)

26. The method of claim 1, further comprising adding at least one adapter to the isolated, captured nucleic acids.

27-30. (canceled)

31. A method of preparing a pooled reporter library for high throughput sequencing, the method comprising:
(a) performing hybrid capture of nucleic acids in a sample comprising a pooled reporter library;
(b) isolating the captured nucleic acids; and
(c) amplifying the isolated, captured nucleic acids.

32. The method of claim 31, wherein the pooled reporter library comprises:
a promoter library, an enhancer library, or a library of regulatory elements; or wherein the pooled reporter library comprises a nucleic acid constant region.

33. (canceled)

34. The method of claim 32, wherein the constant region is a promoter, selectable marker, origin of replication, Cas9 gene, a viral vector backbone, a nucleic acid encoding a fluorescent protein, a nucleic acid encoding a peptide tag, a minimal promoter region, a minimal enhancer region, a minimal splice site region, a minimal 5′ or 3′ untranslated region, or a fragment of each thereof.

35. The method of claim 31, further comprising (d) performing high throughput sequencing analysis of the amplified nucleic acids produced in step (c).

36. A hybridization buffer comprising a buffering agent, a salt, a denaturing agent, and a chelating agent,
wherein the buffering agent is selected from the group of Tris, HEPES, PIPES, PBS, MES, and MOPS;
wherein the salt is selected from the group of NaCl, LiCl, KCl, and NH₄Cl; wherein
the denaturing agent is Urea; and
wherein the chelating agent is selected from the group of EDTA, citric acid, EGTA, and NTA.

37-38. (canceled)
